(19) United States
(12) Patent Application Publication
(10) Pub. No.: US 2010/0168434 A1
(43) Pub. Date: Jul. 1, 2010

(54) INHIBITION OF POLYMERISATION

(75) Inventors: Richard Colin Loyns, Sheffield (GB); Edward David Rippon, Huddersfield (GB); Emyr Phillips, Tingley (GB)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(73) Assignee: A H MARKS AND COMPANY LIMITED, West Yorkshire (GB)

(21) Appl. No.: 12/083,817

(22) PCT Filed: Oct. 19, 2006
(86) PCT No.: PCT/GB2006/003899

§ 371 Date: Mar. 12, 2010

(30) Foreign Application Priority Data
Oct. 20, 2005 (GB) 0521319.4

(51) Int. Cl. C07D 211/94; C07C 253/32; C07C 67/62; C09K 3/00; C07C 13/00

(52) U.S. Cl. 546/242; 558/306; 560/205; 560/261; 560/128; 252/182.29; 585/24

(57) ABSTRACT

The present invention provides methods and compositions for inhibiting polymerisation of ethylenically unsaturated monomers, which involve the use of nitroxide compound of formula (I):

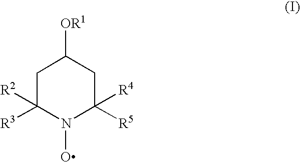

wherein
R¹ is C₄₋₂₀ hydrocarbyl; and
R², R³, R⁴ and R⁵ are independently each C₁₋₆ alkyl.

### FIELD OF THE INVENTION

This invention relates to the inhibition of premature polymerisation of monomers, in particular ethylenically unsaturated monomers.

### BACKGROUND TO THE INVENTION

Many of the industrially important ethylenically unsaturated monomers are highly susceptible to unwanted radical polymerisation. Examples of these monomers include styrene, α-methylstyrene, styrene sulphonic acid, vinyltoluene, divinylbenzenes and dienes such as butadiene or isoprene. Premature polymerisation may occur during manufacture, purification or storage of the monomer. Many of these monomers are purified by distillation. It is in this operation where premature polymerisation is most likely to occur and is the most troublesome. Methods to prevent or reduce the amount of such polymerisations are essential to prevent a dangerous runaway reaction, which can decrease cost-effectiveness of the process and be potentially explosive in nature.

Stable nitroxides are known to inhibit the premature polymerization of ethylenically unsaturated monomers. Many of these nitroxides are based on 2,2,6,6-tetramethylpiperidine-1-oxide (“TEMPO”), a particular example being 4-hydroxy-2,2,6,6-tetramethylpiperidine-1-oxide (“4-hydroxy-TEMPO”), i.e.

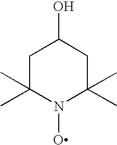

U.S. Pat. No. 5,932,735 and U.S. Pat. No. 6,080,864 describe various TEMPO derivatives which are stated to be useful in inhibiting the premature polymerisation of unsaturated acids, esters, amides, nitriles and ethers; vinyl pyridine, diethyl vinylphosphonate and sodium styrenesulfonate. Examples of the nitroxide compounds taught in this publication include 4-allyloxy-TEMPO and 4-(2-methoxyethoxy)-TEMPO.

U.S. Pat. No. 6,403,850 discloses nitroxide derivatives, including 4-carbomethoxy-TEMPO, 4-carboethoxy-TEMPO and 4-ethanoyloxy-TEMPO, as being useful as inhibitors of premature polymerisation of ethylenically unsaturated monomers.

EP-A-0574666 describes the synthesis of various 4-alkoxy-TEMPO compounds. In particular, this publication teaches the compounds 4-methoxy-TEMPO, 4-ethoxy-TEMPO and 4-n-butoxy-TEMPO.

U.S. Pat. No. 5,631,366 teaches that 4-alkoxy-TEMPO derivatives can be used as catalysts in the synthesis of 3-formylcephem derivatives. Specifically disclosed are 4-methoxy-TEMPO, 4-butoxy-TEMPO and 4-hexoxy-TEMPO.

JP-A-5320217 teaches that 4-alkoxy-TEMPO compounds are useful in preventing polymerisation of methacrylic acid monomers when used in combination with phenothiazines, aromatic amines of phenolic compounds. Specifically disclosed are 4-methoxy-TEMPO and 4-ethoxy-TEMPO.

WO-A-98/56746 describes a composition for inhibiting the premature polymerisation of certain ethylenically unsaturated acrylate monomers. The composition comprises an ethylenically unsaturated acrylate monomer and a synergistic mixture of two nitroxide compounds. This publication describes a range of nitroxide compounds, including 4-hydroxy-TEMPO, 4-propoxy-TEMPO, 4-(2-methoxyethoxyacetoxy)-TEMPO.

The purification of ethylenically unsaturated monomers usually involves the partition of the monomers into organic and aqueous phases. Often, when a stable nitroxide inhibitor is used, the inhibitor only partitions sufficiently well into only one of the phases. Compounds such as 4-hydroxy-TEMPO have good water solubility but are generally poorly soluble in non-polar organic solvents. As a result, premature polymerisation is only effectively inhibited in the aqueous phase, not the organic phase. There remains a need for compounds and compositions which allow for effective inhibition of polymerisation in both the aqueous and organic phases.

### SUMMARY OF THE INVENTION

The present invention is based in part on the discovery that, by using hydrocarbyloxy substituents at the 4-position, the miscibility of TEMPO in organic solvents may be significantly increased. Such derivatives may be effective at inhibiting the premature polymerisation of ethylenically unsaturated monomers, especially when used in combination with a more hydrophilic nitroxide compound such as 4-hydroxy-TEMPO.

According to a first aspect of the invention, a method of inhibiting polymerisation of an ethylenically unsaturated monomer comprises contacting the monomer with a compound of formula (I):

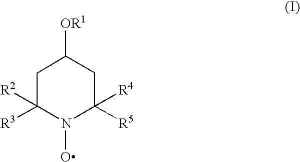

wherein
R¹ is C₄₋₂₀ hydrocarbyl; and
R², R³, R⁴ and R⁵ are independently each C₁₋₆ alkyl.

A second aspect of the invention is a composition for inhibiting polymerisation of an ethylenically unsaturated monomer, which comprises first and second inhibitors of said polymerisation, wherein the first inhibitor is a first nitroxide compound and is of the formula (I), and the second inhibitor is, for example but not limited thereto, a second nitroxide compound.

Another aspect of the invention is the use of a compound of formula (I) as an inhibitor of polymerisation of an ethylenically unsaturated monomer.

Another aspect of the invention is the use of a composition of the invention, as an inhibitor of polymerisation of an ethylenically unsaturated monomer.

The present invention is particularly useful in inhibiting the polymerisation of monomers such as styrenes, vinyltoluenes, divinylbenzenes, dienes (e.g. butadiene or isoprene), acrylonitrile and esters (e.g. butyl acrylate, 2-ethylhexyl acrylate or vinyl acetate). Of particular mention are hydrocarbon monomers. Compounds of the present invention, particularly those wherein R¹ is C₄₋₆ hydrocarbyl, may be prepared in liquid form and thus may be easier and more environmentally acceptable compared with conventional solid inhibitors. Furthermore, the compounds may improve the solubility of monomers and/or other inhibitors in organic solvents.

### DESCRIPTION OF VARIOUS EMBODIMENTS

The term “TEMPO” as used herein refers to 2,2,6,6-tetramethylpiperidine-1-oxide.

The term “ethylenically unsaturated monomer” as used herein refers to a monomer comprising at least one carbon-carbon double bond. Such a monomer may comprise an aliphatic and/or an aromatic moiety. Examples of such monomers include styrenes (e.g. styrene, styrene sulphonic acid and α-methylstyrene), vinyltoluene, divinylbenzenes and dienes (e.g. butadiene and isoprene), esters (including acetates, e.g. vinyl acetate, and acrylates, e.g. 2-ethylhexyl acrylate and butyl acrylate), acids (e.g. methacrylic acid), and the like.

The term “ethylenically unsaturated hydrocarbon monomer” as used herein refers to a monomer comprising at least one carbon-carbon double bond and consisting exclusively of hydrogen and carbon atoms. Such a monomer may comprise an aliphatic and/or an aromatic moiety. Examples of such monomers include styrenes (e.g. styrene and α-methylstyrene), vinyltoluene, divinylbenzenes and dienes (e.g. butadiene and isoprene) and the like.

The term “C₄₋₂₀ hydrocarbyl” refers to a group consisting exclusively of hydrogen and carbon atoms, and which has 4, 5, 6, 7, 8, 9, 10, 11, 12, 13, 14, 15, 16, 17, 18, 19 or 20 carbon atoms. Examples of such groups include C₄₋₁₀ alkyl, C₄₋₁₀ alkenyl, C₄₋₁₀ alkynyl, C₄₋₁₀ cycloalkyl, aryl, —C₁₋₁₀ alkyl-aryl and —C₁₋₁₀ alkyl-C₄₋₁₀ cycloalkyl.

The term “C₁₋₆ alkyl” as used refers to a straight or branched chain alkyl moiety having from 1, 2, 3, 4, 5 or 6 carbon atoms. This term refers to groups such as methyl, ethyl, propyl (n-propyl or isopropyl), butyl (n-butyl, sec-butyl or tert-butyl), pentyl, hexyl and the like.

The term “C₄₋₁₀ alkyl” as used herein refers to a straight or branched chain alkyl moiety having 4, 5, 6, 7, 8, 9 or 10 carbon atoms. This term refers to groups such as butyl (n-butyl, sec-butyl or tert-butyl), pentyl, hexyl, heptyl, octyl, nonyl, decyl and the like.

The term “C₄₋₁₀ alkenyl” as used herein refers to a straight or branched chain alkenyl moiety having 4, 5, 6, 7, 8, 9 or 10 carbon atoms. This term refers to groups such as 1-methylprop-1en-1-yl, 2-methylprop-1-en-1-yl, 1-methylprop-2-en-1-yl, 2-methylprop-2-en-1-yl, penten-1-yl, penten-2-yl, penten-3-yl, penten-4-yl, 1-methylbut-1-en-1-yl, 2-methylbut-1-en-1-yl and the like.

The term “C₄₋₁₀ cycloalkyl” as used herein refers to an alicyclic moiety having 4, 5, 6, 7, 8, 9 or 10 carbon atoms. The group may be a monocyclic, polycyclic, fused or bridged ring system. This term includes reference to groups such as cyclobutyl, cyclopentyl, cyclohexyl, cycloheptyl, cyclooctyl, cyclononyl, cyclodecyl and the like.

The term “aryl” as used herein refers to an aromatic ring system comprising 6, 7, 8, 9, 10, 11, 12, 13, 14, 15 or 16 ring carbon atoms. The group is often phenyl but may be a polycyclic ring system, having two or more rings, at least one of which is aromatic. This term includes reference to groups such as phenyl, naphthyl, fluorenyl and the like.

The term “substituted” as used herein in reference to a moiety or group means that one or more hydrogen atoms in the respective moiety, especially up to 5, more especially 1, 2 or 3 of the hydrogen atoms are replaced independently of each other by the corresponding number of the described substituents. It will, of course, be understood that substituents are only at positions where they are chemically possible, the person skilled in the art being able to decide (either experimentally or theoretically) without inappropriate effort whether a particular substitution is possible. Additionally, it will of course be understood that the substituents described herein may themselves be substituted by any substituent, subject to the aforementioned restriction to appropriate substitutions as recognised by the skilled man.

Embodiments of the invention are described below. It will be appreciated that the features specified in each embodiment may be combined with other specified features, to provide further embodiments.

The present invention involves the use of a compound of formula (I):

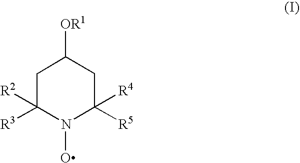

wherein
R¹ is C₄₋₁₀ hydrocarbyl; and
R², R³, R⁴ and R⁵ are independently each C₁₋₆ alkyl.

R², R³, R⁴ and R⁵ may be independently selected from methyl, ethyl, propyl (n-propyl or isopropyl), butyl (n-butyl, sec-butyl or tert-butyl), pentyl and hexyl.

In one embodiment, R², R³, R⁴ and R⁵ are each methyl, i.e. the compound is of the formula (II):

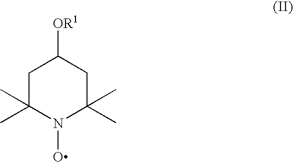

In one embodiment of the invention, R¹ is a C₄₋₁₀ hydrocarbyl group comprising an aliphatic hydrocarbon group (e.g. C₁₋₆ alkyl or C₄₋₁₀ alkyl) optionally substituted with a cyclic hydrocarbon group (e.g. cycloalkyl or aryl).

In another embodiment, R¹ is C₄₋₁₀ alkyl, C₄₋₁₀ alkenyl, aryl or —C₁₋₆ alkyl-aryl, wherein aryl and —C₁₋₆ alkyl-aryl are optionally substituted with C₁₋₆ alkyl or C₂₋₆ alkenyl.

In a further embodiment, R¹ is C₄₋₁₀ alkyl, for example butyl (n-butyl, sec-butyl or tert-butyl), pentyl, hexyl, heptyl, octyl, nonyl or decyl. More preferably, R¹ is n-butyl, sec-butyl or tert-butyl, pentyl, hexyl, heptyl or octyl, in particular n-butyl, sec-butyl or tert-butyl.

In a further embodiment, R¹ is C₄₋₁₀ alkenyl, for example C₄, C₅, C₆, C₇ or C₈ alkenyl. In particular, R¹ may be 1-methylprop-1-en-1-yl, 2-methylprop-1-en-1-yl, 1-methylprop-2-en-1-yl, 2-methylprop-2-en-1-yl, penten-1-yl, penten-2-yl, penten-3-yl, penten-4-yl, 1-methylbut-1-en-1-yl or 2-methylbut-1-en-1-yl.

In a further embodiment, R¹ is aryl, for example phenyl or naphthyl, either of which may be substituted with one or more substituents selected from C₁₋₆ alkyl (e.g. methyl or ethyl), C₂₋₆ alkenyl and C₂₋₆ alkynyl.

In a further embodiment, R¹ is —C₁₋₆ alkyl-aryl optionally substituted with C₁₋₆ alkyl, C₂₋₆ alkenyl or C₂₋₆ alkynyl.

A particular nitroxide compound is 4-butoxy-2,2,6,6-tetramethylpiperidine-1-oxide (“4-butoxy-TEMPO”).

The nitroxide compound may be used in combination with a second inhibitor of polymerisation. The invention therefore provides methods and compositions in which the nitroxide compound is used in combination with a second inhibitor of polymerisation. The second inhibitor is preferably more hydrophilic than the nitroxide compound.

In a particular embodiment, the nitroxide compound is used in combination with a second nitroxide compound which is an inhibitor of premature monomer polymerisation. The second nitroxide compound may be present in a composition comprising the first nitroxide compound.

The second nitroxide compound is preferably more hydrophilic (i.e. more oleophobic, having a greater solubility in aqueous media such as water) than the first nitroxide compound.

In one embodiment, the second nitroxide compound is a compound of formula (III):

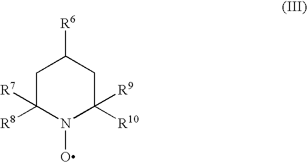

wherein
R⁶ is an atom or group which is more hydrophilic than —OR¹; and
R⁷, R⁸, R⁹ and R¹⁰ are each C₁₋₆ alkyl.

In one embodiment, R⁷, R⁸, R⁹ and R¹⁰ are each independently selected from methyl, ethyl, propyl (n-propyl or isopropyl), butyl (n-butyl, sec-butyl or tert-butyl), pentyl and hexyl.

In another embodiment, R⁷, R⁸, R⁹ and R¹⁰ are each methyl, i.e. the second nitroxide compound is of the formula (IV):

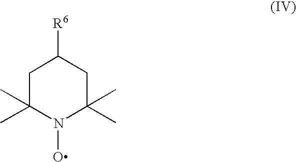

In a particular embodiment, R⁶ is hydroxyl or oxo.

The second nitroxide compound may be 4-hydroxy-2,2,6,6-tetramethylpiperidine-1-oxide (“4-hydroxy-TEMPO”).

Alternatively, the second nitroxide compound may be 4-oxo-2,2,6,6-tetramethylpiperidine-1-oxide (“4-oxo-TEMPO”).

In a preferred embodiment, the first nitroxide compound is 4-butoxy-TEMPO, and the second nitroxide compound is 4-hydroxy-TEMPO.

Alternatively or additionally, the invention may involve the use of one or more inhibitors selected from phenols, alkylated phenols, nitrophenols, nitrosophenols, quinones, hydroquinones, quinone ethers, amines, phenothiazines, hydroxylamines and quinone methides. These compounds may be used in combination with the first nitroxide compound and, in place of or in addition to, the optional second nitroxide compound. Of particular mention are phenothiazine and alkoxylated phenol compounds (e.g. 4-methoxyphenol).

The ethylenically unsaturated monomer may be, for example, a hydrocarbon monomer such as a styrene (e.g. styrene or α-methylstyrene), acrylonitrile, vinyltoluene, a divinylbenzene or a diene (e.g. butadiene or isoprene). Other monomers include esters (e.g. vinyl acetate, butyl acrylate or 2-ethylhexyl acrylate). In a particular embodiment, the monomer is a styrene (e.g. styrene) or acrylonitrile. The monomer may be present in a mixture with one or more comonomers.

A composition of the invention may comprise an ethylenically unsaturated monomer, in particular a hydrocarbon monomer. Again, the ethylenically unsaturated monomer may be a styrene (e.g. styrene, styrene sulphonic acid or α-methylstyrene), acrylonitrile, vinyltoluene, a divinylbenzene, a diene (e.g. butadiene or isoprene), or an ester (e.g.

vinyl acetate, butyl acrylate or 2-ethylhexyl acrylate). In a particular embodiment, the composition comprises a styrene (e.g. styrene) or acrylonitrile. The composition may optionally comprise one or more comonomers.

A composition of the invention may comprise a solvent, e.g. an organic solvent. Of particular mention are non-polar organic solvents, e.g. ethylbenzene.

The amount of the or each nitroxide compound present may be varied according to the conditions and the type of monomer present. For the purposes of illustration, the or each compound may be present in amount of from about 1 to about 1000 ppm (relative to the amount of monomer), more preferably from about 5 to about 500 ppm, more preferably still from about 10 to about 100 ppm. In one embodiment of the invention, the first nitroxide compound is present in an amount of from about 60 to about 80 ppm, and the second nitroxide compound is present in an amount of from about 5 to about 15 ppm.

The inhibitor or inhibitor composition may be brought into contact with monomer by any conventional method. It may be added as a concentrate solution in suitable solvents just upstream of the point of desired application by any suitable means. In addition, these compounds may be injected separately into the extraction or distillation train along with the incoming feed, or through separate entry points providing efficient distribution of the inhibitor composition. Since the inhibitor is gradually depleted during operation, it is generally necessary to maintain the appropriate amount of the inhibitor in the extraction or distillation apparatus by adding inhibitor during the course of the extraction or distillation process. Such addition may be carried out either on a generally continuous basis or it may consist of intermittently charging inhibitor into the extraction or distillation system if the concentration of inhibitor is to be maintained above the minimum required level.

Processes for obtaining the nitroxide compounds of the invention are well known in the art and will be apparent to the skilled person; see, for example, the teachings of EP-A-0574666, U.S. Pat. No. 5,631,366, JP-A-5320217 and WO-A-98/56746. By way of example, the compounds may be obtained by reacting R¹Br with 4-hydroxy-TEMPO in a nucleophilic substitution reaction, conducted in the presence of, for example, aqueous sodium hydroxide, toluene and Bu₄NBr.

The following Examples illustrate the invention.

### Example 1

The solubilities of 4-oxo-TEMPO, 4-hydroxy-TEMPO and 4-butoxy-TEMPO in water and toluene were tested by preparing saturated solutions and measuring the amounts dissolved.

The solubilities of the compounds are given in Table 1. 4-butoxy-TEMPO was found to have a greater solubility in toluene than the 4-oxo- and 4-hydroxy-TEMPO compounds.

TABLE 1

| | Solubility in | |
|---|---|---|
| Compound | water (% wt/wt) | Solubility in toluene (% wt/wt) |
| 4-oxo-TEMPO | 22.5 | >80 |
| 4-hydroxy-TEMPO | 40 | ~10 |
| 4-butoxy-TEMPO | 0.25 | miscible |

### Example 2

The evaluation of the efficacy of a selection of nitroxide compounds of the invention was carried out using a continuous stirred tank reactor (CSTR) which mimicked the re-boiler of a styrene distillation column. The styrene had a residence time of approximately 2 hours inside the reactor and, at 110° C., the CSTR dead volume was 180 ml. A steady state was reached in four hours using a styrene flow rate of 90 ml/hr. Data gathered after this point was averaged to give the steady state polymer level. Nitrogen sparging to remove oxygen gas was carried out at a measured rate of 200 ml/minute. Apart from the inhibitors, the only variable was the inherent variation in the rate of thermal initiation of styrene polymerisation. The nitroxides tested were 4-methoxy-TEMPO, 4-hydroxy-TEMPO, TEMPO, 4-butoxy-TEMPO and 4-allyloxy-TEMPO.

The steady state polymer levels for each compound are given in Table 2. Taking into account differences in molecular weight (for example, the molecular weights of 4-hydroxy-TEMPO and 4-butoxy-TEMPO are 172 and 228 respectively, giving about 25% less 4-butoxy-TEMPO than 4-hydroxy-TEMPO at a fixed ppm level), the inhibitory activity of 4-butoxy-TEMPO is comparable to the inhibitory activities of 4-methoxy-TEMPO and 4-hydroxy-TEMPO.

TABLE 2

| | Concentration | |
|---|---|---|
| Inhibitor | (ppm) | Polymer level at steady state (ppm) |
| 4-methoxy-TEMPO | 75 | 172 |
| 4-hydroxy-TEMPO | 75 | 180 |
| TEMPO | 75 | 213 |
| 4-butoxy-TEMPO | 75 | 341 |
| 4-allyloxy-TEMPO | 75 | 15199 (at 120 mins) |
| No inhibitor | 0 | >30000 |

### Example 3

The experiment of Example 2 was repeated using combinations of 4-hydroxy-TEMPO and the other compounds.

The steady state polymer levels for each combination are given in Table 3. It is evident that the combination of 4-butoxy-TEMPO and 4-hydroxy-TEMPO was a more effective inhibitor than any of the other combinations. In addition, this combination was considerably more effective than the individual compounds as tested in Example 2.

TABLE 3

| | | Polymer level |
|---|---|---|
| | Concentration | at steady |
| Inhibitor | (ppm) | state (ppm) |
| 4-butoxy-TEMPO/4-hydroxy-TEMPO | 67.7/7.5 | 68 |
| 4-methoxy-TEMPO/4-hydroxy-TEMPO | 67.7/7.5 | 169 |
| TEMPO/4-hydroxy-TEMPO | 67.7/7.5 | 239 |
| 4-allyloxy-TEMPO/4-hydroxy-TEMPO | 67.7/7.5 | 12291 |
| | | (at 210 mins) |

### Example 4

The efficacy of various inhibitors was evaluated with respect to the monomers vinyl acetate, acrylonitrile, 2-ethylhexyl acrylate and isoprene. The efficacy of each inhibitor was determined by heating in pure monomer or a solution of monomer in a suitable solvent(s) for a set time and at a known temperature. The storage inhibitor was removed from each monomer by distillation or treatment with neutral silica. In each of the tables below, the amount of polymer generated is expressed as % w/w of the monomer.

### Vinyl Acetate Monomer

In this case, the tubes and solution were degassed with nitrogen prior to sealing and heating at 160° C. for 48 hours.

As the table below illustrates, 4-butoxy-TEMPO was found to be a more effective inhibitor than phenothiazine. Synergy was observed with a combination of 4-butoxytempo and phenothiazine.

| Inhibitor | Concentration (ppm) | Polymer (% w/w) |
|---|---|---|
| Blank | — | 100 (Completely solid) |
| 4-Butoxy-TEMPO | 25 | 19 |
| Phenothiazine | 25 | 35 |
| 4-Butoxy-TEMPO/ | 12.5/12.5 | 5 |
| Phenothiazine | | |

### Acrylonitrile Monomer

Similarly, tubes containing acrylonitrile and various inhibitors were heated at 120° C. for 7 days. The air was not removed from these solutions prior to heating.

As the table below illustrates, 4-butoxy-TEMPO was found to be a more effective inhibitor than 4-hydroxy-TEMPO.

| Inhibitor | Concentration (ppm) | Polymer (% w/w) |
|---|---|---|
| Blank | — | 1.28 |
| 4-Butoxytempo | 10 | 0.06 |
| 4-Hydroxytempo | 10 | 0.18 |

### 2-Ethylhexyl Acrylate Monomer

Similarly, tubes containing 5 ppm inhibitor in 2-ethylhexyl acrylate were heated at 145° C. and the results expressed as inhibition time (i.e. the time to appearance of insoluble polymer). Data were generated in nitrogen and in the presence of air. As the table below illustrates, 4-butoxy-TEMPO was found to be an effective inhibitor of polymerisation.

| | Inhibition | Inhibition |
|---|---|---|
| Inhibitor | time in air (min) | time in nitrogen (min) |
| Blank | 15 | 55 |
| 4-Butoxytempo | 40 | 350 |

### Isoprene Monomer

Similarly tubes containing 100 ppm inhibitor in a solution of Isoprene in dimethylformamide containing 1 or 3% furfural were heated at 160° C. for 1 hr. As the table below illustrates, 4-butoxy-TEMPO was found to be an effective inhibitor of polymerisation. The results are expressed as ppm insoluble polymer.

| Inhibitor | 1% Furfural | 3% Furfural |
|---|---|---|
| Blank | 8.7 | 13.7 |
| 4-Butoxytempo | 2.2 | 5.3 |

### Example 5

Saturated solutions of the inhibitors/retarders phenothiazine and 4-methoxyphenol were prepared in 4-butoxy-TEMPO. These saturated solutions were then added to styrene so that the concentration of each inhibitor/retarder was above the solubility limit for that material in styrene alone. The solutions were observed for physical stability at 20° C. The table below shows the soluble amount of each inhibitor/retarder in 4-butoxy-TEMPO plus styrene:

| | Wt % test material (with | |
|---|---|---|
| Test Material | respect to styrene) | Appearance |
| Phenothiazine | 7.0 | Clear solution |
| 4-Methoxyphenol | 32.4 | Clear solution |

The same inhibitors were then added to styrene monomer at various concentrations in the absence of 4-butoxy-TEMPO, to determine if solubility was enhanced by including 4-butoxyTEMPO in the formulation. The table below shows the solubility of the inhibitors/retarders in styrene in the absence of 4-butoxy-TEMPO:

| | Wt % test material (with | |
|---|---|---|
| Test Material | respect to styrene) | Appearance |
| Phenothiazine | 7.0 | Insoluble |
| 4-Methoxyphenol | 32.4 | Insoluble |

Comparing the two sets of experiments, it was apparent that the presence of 4-butoxy-TEMPO enhanced the solubility of the other inhibitors.

### Example 6

The solubility of 4-hydroxytempo in ethylbenzene is only 8.1% wt/wt at 20° C. A solution of 4-hydroxy-TEMPO and 4-butoxy-TEMPO in ethylbenzene was prepared with a 4-hydroxy-TEMPO concentration of 14.9% with respect to ethylbenzene. This gave a clear red solution, indicating that the presence of 4-butoxy-TEMPO enhanced the solubility of 4-hydroxy-TEMPO in ethylbenzene.

1. (canceled)

2. (canceled)

3. (canceled)

4. (canceled)

5. (canceled)

6. (canceled)

7. A method of inhibiting polymerization of an ethylenically unsaturated monomer, which comprises contacting the monomer with 4-butoxy-2,2,6,6-tetramethylpiperdine-1-oxide.

8. The method according to claim 7, which further comprises contacting the monomer with a second inhibitor of said polymerisation.

9. The method according to claim 8, wherein the second inhibitor is a second nitroxide compound.

10. The method according to claim 9, wherein the second nitroxide compound is more hydrophilic than the first nitroxide compound.

11. The method according to claim 10, wherein the second nitroxide compound is a compound of formula (III): 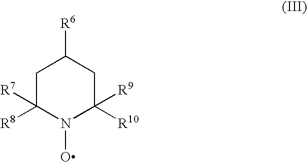
wherein
R⁶ is an atom or group which is more hydrophilic than —OR¹; and
R⁷, R⁸, R⁹ and R¹⁰ are each C₁₋₆ alkyl.

12. The method according to claim 11, which is of formula (IV):

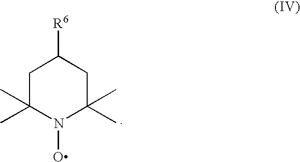

13. The method according to claim 11, wherein R⁶ is hydroxy or oxo.

14. (canceled)

15. (canceled)

16. (canceled)

17. (canceled)

18. (canceled)

19. The method according to claim 8, wherein the second inhibitor is selected from phenols, alkylated phenols, nitrophenols, nitrosophenols, quinones, hydroquinones, quinone ethers, amines, phenothiazines, hydroxylamines and quinone methides.

20. (canceled)

21. The method according to claim 7, wherein the monomer is a hydrocarbon monomer.

22. The method according to claim 21, wherein the monomer is selected from the group consisting of styrene, α-methylstyrene, acrylonitrile, vinyltoluene, a divinylbenzene and a diene.

23. (canceled)

24. The method according to claim 7, wherein the monomer is an ester.

25. The method according to claim 24, wherein the monomer is selected from the group consisting of butyl acrylate, vinyl acetate and 2-ethylhexyl acrylate.

26. (canceled)

27. (canceled)

28. (canceled)

29. (canceled)

30. (canceled)

31. (canceled)

32. A composition for inhibiting polymerization of an ethylenically unsaturated monomer, which comprises a first inhibitor and a second inhibitor of polymerization, wherein the first monomer is 4-butoxy-2,2,6,6-tetramethyl piperdine-1-oxide.

33. The composition according to claim 32, wherein the second inhibitor is a second nitroxide compound.

34. (canceled)

35. The composition according to claim 33, wherein the second nitroxide compound is a compound of formula (III): 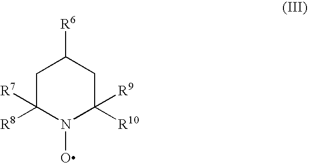
wherein
R⁶ is an atom or group which is more hydrophilic than —OR¹; and
R⁷, R⁸, R⁹ and R¹⁰ are each C₁₋₆ alkyl.

36. The composition according to claim 35, wherein the second nitroxide compound is of formula (IV):

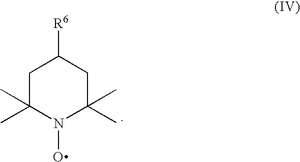

37. The composition according to claim 35, wherein R⁶ is hydroxy or oxo.

38. (canceled)

39. (canceled)

40. (canceled)

41. (canceled)

42. (canceled)

43. The composition according to claim 32, wherein the second inhibitor is selected from phenols, alkylated phenols, nitrophenols, nitrosophenols, quinones, hydroquinones, quinone ethers, amines, phenothiazines, hydroxylamines and quinone methides.

44. (canceled)

45. The composition according to claim 32, which further comprises an ethylenically unsaturated monomer.

46. The composition according to claim 45, wherein the monomer is selected from the group consisting of styrene, α-methylstyrene, acrylonitrile, vinyltoluene, a divinylbenzene and a diene.

47. (canceled)

48. (canceled)

49. The composition according to claim 45, wherein the monomer is an ester.

50. The composition according to claim 49, wherein the monomer is selected from the group consisting of butyl acrylate, vinyl acetate and 2-ethylhexyl acrylate.

51. An inhibitor of polymerisation of an ethylenically unsaturated monomer comprising 4-butoxy-2,2,6,6-tetramethylpiperidine-1-oxide.

52. (canceled)

53. (canceled)
